(19) United States
(12) Patent Application Publication
(10) Pub. No.: US 2021/0060017 A1
(43) Pub. Date: Mar. 4, 2021

(54) METHOTREXATE OPHTHALMIC SOLUTION

(71) Applicant: SHILPA MEDICARE LIMITED, RAICHUR (IN)

(72) Inventors: ANDANAYYA SARGANACHARI, Bangalore (IN); SANDEEP SUKUMAR, Bangalore (IN); SREENIVASA REDDY, Bangalore (IN); PRADEEP SHIVAKUMAR, Vizianagaram (IN); THIPPESWAMY AHM, Bangalore (IN)

(73) Assignee: SHILPA MEDICARE LTD, RAICHUR (IN)

(21) Appl. No.: 16/960,090

(22) PCT Filed: Feb. 22, 2019
(86) PCT No.: PCT/IB2019/051447

§ 371 Date: Jul. 6, 2020

(30) Foreign Application Priority Data
Feb. 27, 2018 (IN) 201814007303

(51) Int. Cl. A61K 31/519; A61K 9/00; A61K 9/08; A61K 47/18; A61K 47/36; A61P 27/02

(52) U.S. Cl. CPC A61K 31/519; A61K 9/0048; A61P 27/02; A61K 47/18; A61K 47/36; A61K 9/08

(57) ABSTRACT

The present invention relates to a topical ophthalmic composition formulated for application to the eye, said composition comprising a therapeutically effective amount of methotrexate for the treatment of anterior uveitis. Preferably anterior uveitis is auto immune mediated and non-infectious. Further the present invention relates to a method of treating anterior uveitis of the mammalian eye, said method comprising administering to the mammal in eye about 5 μg to about 200 μg of methotrexate.

### FIELD OF THE INVENTION

The present invention relates to ophthalmic formulations; more particularly to ophthalmic formulations of methotrexate; most particularly eye drops of methotrexate used for the treatment of anterior uveitis. The anterior uveitis preferably is auto immune mediated and non-infectious acute anterior uveitis.

### BACKGROUND OF THE INVENTION

Methotrexate is structurally represented as

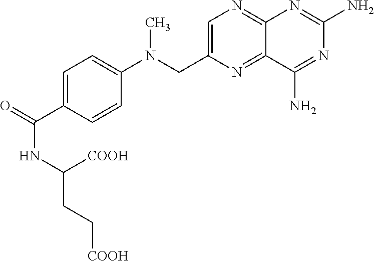 [SMILES: CN(Cc1cnc2nc(N)nc(N)c2n1)c1ccc(C(=O)NC(CCC(=O)O)C(=O)O)cc1]

Methotrexate based medicaments have been known in the art for years, and have been shown to possess anti-neoplastic, anti-angiogenic, anti-fibroblastic, and/or immunosuppressive activities. While administering methotrexate based medicaments have been identified as a promising remedy to treat many of the above-identified irregularities, delivering methotrexate based medicaments to an affected area of a living subject's eye has remained heretofore largely problematic. For example, delivering methotrexate based medicaments to an affected, local area of a living subject's eye using a systemic delivery method is problematic because of the many severe, sometimes life threatening, side effects associated with systemic delivery of methotrexate based medicaments, such as, for examples, hepatitis, liver fibrosis, cirrhosis, leukopenia (bone marrow suppression), mucositis, ulcerative stomatitis, skin rash, nausea, abdominal distress, malaise, fatigue, chills and fever, diarrhea, gastrointestinal ulceration or perforation, pancreatitis, pericarditis, hypotension, deep venous thrombosis, thrombophlebitis, interstitial pneumonitis, headaches, drowsiness, cognitive dysfunction, reduced immunity, rash, photosensitivity, nephropathy, hematuria, alopecia, defective oogenesis, oligospermia, infertility, miscarriage, and birth defects.

Munoz-Fernandez et al; Eye (2009) 23, 1130-1133; discloses the usage of oral methotrexate as an option for preventing the recurrence of anterior uveitis.

Malik et al; Br J Ophthalmol 2005; 89:806-808; discloses the usage of combinations of oral and subcutaneous low dose usage of methotrexate with oral or topical corticosteroids for treatment of chronic anterior and intermediate uveitis.

PCT Patent Publication No. WO2016019165 discloses the method of treating or reducing the risk of proliferative vitreoretinopathy (PVR) or epiretina membranes (ERM) in a subject with intravitreal injections of methotrexate.

The above prior art suggests the usage of low dose oral methotrexate for the treatment of anterior uveitis with systemic side effects and does not suggest the ophthalmic administration of methotrexate for anterior uveitis. Therefore, there exists a need to develop the ophthalmic solution comprising methotrexate for treatment of anterior uveitis to overcome the systemic side effects of methotrexate.

### SUMMARY OF THE INVENTION

In one object, the present invention relates to a topical ophthalmic composition formulated for application to the eye, said composition comprising a therapeutically effective amount of methotrexate for the treatment of anterior uveitis. Preferably anterior uveitis is auto immune mediated and non-infectious.

In another object, the present invention relates to a method of treating anterior uveitis of the mammalian eye, said method comprising administering to the mammal in eye about 5 μg to about 200 μg of methotrexate.

In a further object, the present invention relates to a method of treating anterior uveitis of the mammalian eye, said method comprising administering to the mammal in eye about 5 μg to about 200 μg of methotrexate, wherein methotrexate is administered as an ophthalmic solution once a day, twice a day or thrice a day in each affected eye.

In another object, the present invention relates to a topical ophthalmic composition formulated for application to the eye, said composition comprising a therapeutically effective amount of methotrexate, solubilizers, viscosifying agents, buffering agents, osmotic agents, antioxidants, pH adjusting agents, optionally preservatives and water for injection.

In a still further object, the present invention relates to a topical ophthalmic composition formulated for application to the eye, said composition comprising a therapeutically effective amount of methotrexate, tromethamine, xanthan gum, trisodium citrate, sodium chloride, disodium EDTA and pH adjusting agents, wherein the composition has the pH of about 6.8 to about 8.5.

### BRIEF DESCRIPTION OF THE DRAWINGS

FIG. 1 discloses effect of methotrexate formulations of examples 1, 2 and 3 for treatment of anterior uveitis in rabbit's eye after 24 hours of intra-vitreal BSA injection.

FIG. 2 discloses effect of methotrexate formulations of examples 5, 6, 7, 8 and 9 for treatment of anterior uveitis in rabbit's eye after 24 hours of intra-vitreal BSA injection.

FIG. 3 discloses effect of methotrexate formulations of examples 5, 6, 7, 8 and 9 for treatment of anterior uveitis in rabbit's eye after 48 hours of intra-vitreal BSA injection.

FIG. 4 discloses effect of methotrexate formulations of examples 5, 6, 7, 8 and 9 for treatment of anterior uveitis in rabbit's eye after 72 hours of intra-vitreal BSA injection.

### DEFINITION OF SELECTED TERMS

In describing and claiming the present invention, the following terminologies will be used in accordance with the definitions set out below.

The term “Uveitis” swelling of the middle layer of the eye, which is called the uvea. It may occur from both infectious and non-infectious causes. The uvea supplies blood to the retina. The retina is the light-sensitive part of the eye that focuses the images you see and sends them to the brain. It's normally red due to its blood supply from the uvea.

Anterior uveitis is an inflammation of the middle layer of the eye. This layer includes the iris (colored part of the eye) and the adjacent tissue, known as the ciliary body. If untreated, it can cause permanent damage and loss of vision from the development of glaucoma, cataract or retinal edema.

The term “ophthalmic solution” a sterile solution that is free from foreign particles and is compounded and dispensed for eye drops.

As used herein in connection with numerical values, the terms “about” mean+/−10% of the indicated value, including the indicated value.

The term “therapeutically effective amount of methotrexate” means about 5 μg to about 200 μg of methotrexate administered into the mammal's each eye once, twice, thrice or four times a day.

### DETAILED DESCRIPTION OF THE INVENTION

The present invention generally provides topical ophthalmic composition formulated for application to the eye, said composition comprising a therapeutically effective amount of methotrexate for the treatment of anterior uveitis. Preferably anterior uveitis is auto immune mediated and non-infectious.

The present invention generally provides topical ophthalmic composition, preferably in the form of solution, suspensions or emulsions for application to the eye, said composition comprising a therapeutically effective amount of methotrexate for the treatment of anterior uveitis.

In one embodiment, the present invention provides a method of treating anterior uveitis of the mammalian eye, said method comprising administering to the mammal in eye about 5 μg to about 200 μg of methotrexate.

In another embodiment, the present invention provides a method of treating anterior uveitis of the mammalian eye, said method comprising administering to the mammal in eye about 5 μg to about 200 μg of methotrexate, wherein methotrexate is administered as an ophthalmic solution once a day, twice a day or thrice a day in each affected eye.

In embodiments of the present invention, methotrexate is administered to the mammalian eye in the dosages of about 5 μg, 7.5 μg, 10 μg, 12.5 μg, 15 μg, 17.5 μg, 20.0 μg, 22.5 μg, 25.0 μg, 27.5 μg, 30.0 μg, 32.5 μg, 35.0 μg, 37.5 μg, 40.0 μg, 42.5 μg, 45.0 μg, 47.5 μg, 50 μg, 52.5 μg, 55 μg, 57.5 μg, 60 μg, 62.5 μg, 65 μg, 67.5 μg, 70 μg, 72.5 μg, 75 μg, 77.5 μg, 80 μg, 82.5 μg, 85 μg, 87.5 μg, 90 μg, 92.5 μg, 95 μg, 97.5 μg, 100 μg, 102.5 μg, 105 μg, 107.5 μg, 10 μg, 112.5 μg, 115 μg, 117.5 μg, 120 μg, 122.5 μg, 125 μg, 127.5 μg, 130 μg, 132.5 μg, 135 μg, 137.5 μg, 140 μg, 142.5 μg, 145 μg, 147.5 μg, 150 μg, 152.5 μg, 155 μg, 157.5 μg, 160 μg, 162.5 μg, 165 μg, 167.5 μg, 170 μg, 172.5 μg, 175 μg, 177.5 μg, 180 μg, 182.5 μg, 185 μg, 187.5 μg, 190 μg, 192.5 μg, 195 μg, 197.5 μg and 200 μg in each affected eye once a day, twice a day, thrice a day or four times a day.

In another embodiment, the present invention provides a topical ophthalmic composition formulated for application to the eye, said composition comprising a therapeutically effective amount of methotrexate, solubilizers, viscosifying agents, buffering agents, osmotic agents, antioxidants, pH adjusting agents, optionally preservatives and purified water.

In embodiments of the invention, the present invention provides a method of treating anterior uveitis of the mammalian eye, said method comprising administering to the mammal in eye a methotrexate topical ophthalmic composition comprising therapeutically effective amount of methotrexate, solubilizers, viscosifying agents, buffering agents, osmotic agents, antioxidants, pH adjusting agents, optionally preservatives and purified water.

In embodiments of the invention, the composition preferably comprises about 0.01% w/v to about 0.5% w/v methotrexate; more preferably 0.01% w/v to about 0.1% w/v methotrexate; even more preferably 0.01% w/v, 0.015% w/v, 0.02% w/v, 0.025% w/v, 0.03% w/v, 0.035% w/v, 0.04% w/v, 0.045% w/v, 0.05% w/v, 0.055% w/v, 0.06% w/v, 0.065% w/v, 0.07% w/v, 0.075% w/v, 0.08% w/v, 0.085% w/v, 0.09% w/v, 0.095% w/v, 0.1% w/v methotrexate, and most preferably 0.025% w/v of methotrexate.

In embodiments of the invention, the composition comprises solubilizers selected from the group consisting of tromethamine, pluronics, polyethylene glycol, polysorbate 20, polysorbate 80, lecithin, hydroxypropyl β-cyclodextrin, cholesterol and tyloxapol. Most preferred solubilizer used in the present invention is tromethamine. The amount of solubilizers ranges from about 0.1% w/v to about 1.0% w/v, more preferably in the range from about 0.2% w/v to about 0.8% w/v.

The amount of tromethamine present in the composition ranges from about 0.1% w/v to about 1.0% w/v, more preferably 0.2% w/v to about 0.8% w/v and most preferably 0.4% w/v. Other preferred percentages of tromethamine include 0.1% w/v, 0.15% w/v, 0.2% w/v, 0.25% w/v, 0.3% w/v, 0.35% w/v, 0.4% w/v, 0.45% w/v, 0.5% w/v, 0.55% w/v, 0.6% w/v, 0.65% w/v, 0.7% w/v, 0.75% w/v, 0.8% w/v, 0.85% w/v and 0.9% w/v. It has been surprisingly found by the inventors that the composition comprising of about 0.2% w/v to about 0.8% w/v has total impurities less than 2.0% and individual impurity less than 1.0% after storage of composition for six months at 2-8° C.

An impurity level of the methotrexate ophthalmic solution is determined by use of high performance liquid chromatography-mass spectrometry (HPLC-MS or LC-MS). For long term storage, the methotrexate ophthalmic compositions are typically stored under a refrigerated condition (2 to 8° C.) with very low humidity. In some embodiments, methotrexate total degradation impurities in the final product are less than about 2.0% under the refrigerated storage condition for six months and the individual impurity is less than 1.0%.

In embodiments of the invention, the composition comprises viscosifying agents selected from the group consisting of polyvinyl alcohol and derivatives, polyvinylpyrrolidone and derivatives, hydroxypropylmethylcellulose and derivatives, carboxymethylcellulose and its derivatives, tamarind seed polysaccharide (xyloglucan), polyacrylic acids, xanthan gum and its derivatives, beta glucan and its derivatives. The most preferred viscosifying agent is xanthan gum. The amount of viscosifying agent ranges from about 0.1% w/v to about 1.0% w/v, more preferably in the range from about 0.2% w/v to about 0.8% w/v and most 30 preferably 0.6% w/v. The amount of xanthan gum present in the composition ranges from about 0.1% w/v to about 1.0% w/v, more preferably 0.2% w/v to about 0.8% w/v and most preferably 0.6% w/v. Other preferred percentages of xanthan gum include 0.1% w/v, 0.15% w/v, 0.2% w/v, 0.25% w/v, 0.3% w/v, 0.35% w/v, 0.4% w/v, 0.45% w/v, 0.5% w/v, 0.55% w/v, 0.6% w/v, 0.65% w/v, 0.7% w/v, 0.75% w/v, 0.8% w/v, 0.85% w/v and 0.9% w/v.

In embodiments of the invention, the composition comprises buffering agents selected from the group consisting of one or more citrate salt(s) and/or the free acid thereof (for example citric acid, citric acid monohydrate, trisodium citrate dihydrate, tripotassium citrate monohydrate), acetate salt(s) and/or the free acid thereof (for example acetic acid, sodium acetate, sodium acetate trihydrate) or L-histidine and/or an acid-addition salt thereof, such as, for example, L-histidine monohydrochloride monohydrate. Preferably buffering agent selected is trisodium citrate dihydrate. The amount of buffering agent in the present composition ranges from about 0.05% w/v to about 0.5% w/v and most preferably from about 0.1% w/v to about 0.3% w/v.

In embodiments of the invention, the composition comprises osmotic agents selected from the group consisting of trehalose, mannitol, sorbitol, lactulose, sodium chloride, and propylene glycol. Preferably osmotic agent selected is sodium chloride. The amount of osmotic agent in the present composition ranges from about 0.1% w/v to about 1% w/v and most preferably from about 0.3% w/v to about 0.8% w/v.

In embodiments of the invention, the composition comprises antioxidants selected from the group consisting of butylated hydroxyanisole (BHA), butylated hydroxytoluene (BHT), ascorbic acid, beta-carotene, alpha-tocopherol, propyl gallate, gentisic acid sodium ascorbate, sodium bisulfite, sodium metabisulfite, monothioglycerol, cysteine, thioglycolate sodium, acetone sodium bisulfite, ascorbate (sodium/acid), bisulfite sodium, cystein/cysteinate HCl, dithionite sodium (Na hydrosulfite, Na sulfoxylate), gentisic acid, gentisic acid ethanolamine, glutamate monosodium, formaldehyde sulfoxylate sodium, metabisulfite potassium, metabisulfite sodium, monothioglycerol (thioglycerol), propyl gallate, sulfite sodium, tocopherol alpha, thioglycolate sodium, EDTA in calcium and sodium compounds (disodium EDTA) or the mixtures thereof. Preferably antioxidant selected is disodium EDTA. The amount of antioxidant in the present composition ranges from about 0.005% w/v to about 0.5% w/v and most preferably from about 0.01% w/v to about 0.05% w/v.

In embodiments of the invention, the composition comprises pH adjusting agents selected from the group consisting of consisting of sodium bicarbonate, sodium carbonate, sodium hydroxide, potassium carbonate, potassium carbonate, calcium carbonate, amino acid derivatives, alkali metal carbonate (such as sodium glycine carbonate) hydrochloric acid, nicotinic acid, acetylsalicylic acid, adipic acid or mixtures thereof. Preferably, the pH adjusting agents are selected from sodium hydroxide and hydrochloride acid. pH adjusting agents are used in the present composition to adjust the pH from about 6.8 to about 8.5. The inventors of the present invention have surprisingly found that ophthalmic composition of methotrexate are stable to form a clear solution in the pH range from about 6.8 to about 8.5; below the pH range of about 6.8, and above pH range of about 8.5, the present inventors have observed that the composition is turbid and does not form a clear solution that is administrable into the eye.

In embodiments of the invention, the composition comprises preservative selected from the group consisting of benzalkonium chloride (BAK), butyl paraben and propyl paraben. Most preferred preservative used in the present invention is BAK.

In embodiments of the invention, the present invention provides the topical ophthalmic composition formulated for application to the eye, said composition comprises about 0.01% w/v to about 0.05% w/v of methotrexate, about 0.2% w/v to about 0.8% w/v of solubilizers, about 0.1% w/v to about 1.0% w/v of viscosifying agents and pH adjusting agents; wherein the composition has the pH of about 6.8 to about 8.5.

In another embodiment of the invention, the present invention provides the topical ophthalmic composition formulated for application to the eye, said composition consisting essentially of about 0.01% w/v to about 0.05% w/v of methotrexate, about 0.2% w/v to about 0.8% w/v of solubilizers, about 0.1% w/v to about 1.0% w/v of viscosifying agents, buffering agents, osmotic agents, antioxidants, preservatives and pH adjusting agents; wherein the composition has the pH of about 6.8 to about 8.5.

In further embodiment of the invention, the present invention provides the topical ophthalmic composition formulated for application to the eye, said composition consisting of about 0.01% w/v to about 0.05% w/v of methotrexate, about 0.2% w/v to about 0.8% w/v of solubilizers, about 0.1% w/v to about 1.0% w/v of viscosifying agents, buffering agents, osmotic agents, antioxidants, preservatives and pH adjusting agents; wherein the composition has the pH of about 6.8 to about 8.5.

In embodiments of the invention, the present invention provides the topical ophthalmic composition formulated for application to the eye, said composition comprises about 0.01% w/v to about 0.05% w/v of methotrexate, about 0.2% w/v to about 0.8% w/v of tromethamine, about 0.1% w/v to about 1.0% w/v of xanthan gum and pH adjusting agents; wherein the composition has the pH of about 6.8 to about 8.5.

In another embodiment of the invention, the present invention provides the topical ophthalmic composition formulated for application to the eye, said composition consisting essentially of about 0.01% w/v to about 0.05% w/v of methotrexate, about 0.2% w/v to about 0.8% w/v of tromethamine, about 0.1% w/v to about 1.0% w/v of xanthan gum, buffering agents, osmotic agents, antioxidants, preservatives and pH adjusting agents; wherein the composition has the pH of about 6.8 to about 8.5.

In a further embodiment of the invention, the present invention provides the topical ophthalmic composition formulated for application to the eye, said composition consisting of about 0.01% w/v to about 0.05% w/v of methotrexate, about 0.2% w/v to about 0.8% w/v of tromethamine, about 0.1% w/v to about 1.0% w/v of xanthan gum, buffering agents, osmotic agents, antioxidants, preservatives and pH adjusting agents; wherein the composition has the pH of about 6.8 to about 8.5.

In embodiments of the invention, the present invention provides the topical ophthalmic composition formulated for application to the eye, said composition comprises about 0.025% w/v of methotrexate, about 0.4% w/v of tromethamine, about 0.6% w/v of xanthan gum and pH adjusting agents; wherein the composition has the pH of about 6.8 to about 8.5.

In another embodiment of the invention, the present invention provides the topical ophthalmic composition formulated for application to the eye, said composition consisting essentially of about 0.025% w/v of methotrexate, about 0.4% w/v of tromethamine, about 0.6% w/v of xanthan gum, buffering agents, osmotic agents, antioxidants, preservatives and pH adjusting agents; wherein the composition has the pH of about 6.8 to about 8.5.

In further embodiment of the invention, the present invention provides the topical ophthalmic composition formulated for application to the eye, said composition consisting of about 0.025% w/v of methotrexate, about 0.4% w/v of tromethamine, about 0.6% w/v of xanthan gum, buffering agents, osmotic agents, antioxidants, preservatives and pH adjusting agents; wherein the composition has the pH of about 6.8 to about 8.5.

In embodiments of the invention, the present invention provides the topical ophthalmic composition formulated for application to the eye, said composition comprises about 0.01% w/v to about 0.05% w/v of methotrexate, about 0.2% w/v to about 0.8% w/v of tromethamine, about 0.1% w/v to about 1.0% w/v of xanthan gum and pH adjusting agents; wherein the composition has the pH of about 6.8 to about 8.5 and wherein an individual impurity present in the said composition is less than 1.0% and total amount of impurities present in the said composition is less than 2.0%.

In another embodiment of the invention, the present invention provides the topical ophthalmic composition formulated for application to the eye, said composition consisting essentially of about 0.01% w/v to about 0.05% w/v of methotrexate, about 0.2% w/v to about 0.8% w/v of tromethamine, about 0.1% w/v to about 1.0% w/v of xanthan gum, buffering agents, osmotic agents, antioxidants, preservatives and pH adjusting agents; wherein the composition has the pH of about 6.8 to about 8.5 and wherein an individual impurity present in the said composition is less than 1.0% and total amount of impurities present in the said composition is less than 2.0%.

In a further embodiment of the invention, the present invention provides the topical ophthalmic composition formulated for application to the eye, said composition consisting of about 0.01% w/v to about 0.05% w/v of methotrexate, about 0.2% w/v to about 0.8% w/v of tromethamine, about 0.1% w/v to about 1.0% w/v of xanthan gum, buffering agents, osmotic agents, antioxidants, preservatives and pH adjusting agents; wherein the composition has the pH of about 6.8 to about 8.5 and wherein an individual impurity present in the said composition is less than 1.0% and total amount of impurities present in the said composition is less than 2.0%.

In embodiments of the invention, the present invention provides the topical ophthalmic composition formulated for application to the eye, said composition comprises about 0.025% w/v of methotrexate, about 0.4% w/v of tromethamine, about 0.6% w/v of xanthan gum and pH adjusting agents; wherein the composition has the pH of about 6.8 to about 8.5 and wherein an individual impurity present in the said composition is less than 1.0% and total amount of impurities present in the said composition is less than 2.0%.

In another embodiment of the invention, the present invention provides the topical ophthalmic composition formulated for application to the eye, said composition consisting essentially of about 0.025% w/v of methotrexate, about 0.4% w/v of tromethamine, about 0.6% w/v of xanthan gum, buffering agents, osmotic agents, antioxidants, preservatives and pH adjusting agents; wherein the composition has the pH of about 6.8 to about 8.5 and wherein an individual impurity present in the said composition is less than 1.0% and total amount of impurities present in the said composition is less than 2.0%.

In further embodiment of the invention, the present invention provides the topical ophthalmic composition formulated for application to the eye, said composition consisting of about 0.025% w/v of methotrexate, about 0.4% w/v of tromethamine, about 0.6% w/v of xanthan gum, buffering agents, osmotic agents, antioxidants, preservatives and pH adjusting agents; wherein the composition has the pH of about 6.8 to about 8.5 and wherein an individual impurity present in the said composition is less than 1.0% and total amount of impurities present in the said composition is less than 2.0%.

The following examples are provided to illustrate the present invention. It is understood, however, that the invention is not limited to the specific conditions or details described in the examples below. The examples should not be construed as limiting the invention as the examples merely provide specific methodology useful in the understanding and practice of the invention and its various aspects. While certain preferred and alternative embodiments of the invention have been set forth for purposes of disclosing the invention, modification to the disclosed embodiments can occur to those who are skilled in the art.

### Examples 1 & 2: Ophthalmic Solutions of Methotrxate

Composition:

| | Example 1 | Example 2 |
|---|---|---|
| Name of Ingredient | % w/v | % w/v |
| Methotrexate | 0.1% | 0.5% |
| Tromethamine | 0.4% | 0.4% |
| Xanthan gum | 0.6% | 0.6% |
| Trisodium citrate dihydrate | 0.2% | 0.2% |
| Sodium chloride | 0.68% | 0.68% |
| Disodium EDTA | 0.025% | 0.025% |
| Sodium hydroxide/ | Qs to adjust | Qs to |
| hydrochloric acid | pH 7.2 | pH 7.2 |
| Purified water | Qs | Qs |

Process for Preparation: These ophthalmic solution is prepared as follows:
1.1. Collect 100% batch size of purified water, purge with Nitrogen, Dissolved oxygen content should be less than 2 PPM, check pH.

Preparation of Polymer Phase:
1.1.1. Collect 75% total batch size of purified water in a clean SS vessel, maintain purified water temperature at 70° C., under stirring add dispensed quantity of xanthan gum, after ensuring complete solubilisation, allow the polymeric solution to attain room temperature. Make up the volume to 80% of batch size using purified water from step 1.1.
1.1.2. Filter step 1.1.1 polymeric solution.
1.1.3. Autoclave the polymer solution at 121° C. for 15 min, allow to attain 20-25° C.

Preparation of API Phase:
1.1.4. Collect 15% total batch size of purified water in a clean SS vessel from step 1.1.
1.1.5. Under stirring, add dispensed quantity of Tromethamine to step 1.1.4 purified water, ensure complete solubilisation.
1.1.6. Under stirring, add dispensed quantity of Methotrexate to step 1.1.5 purified water, ensure complete solubilisation.
1.1.7. Under stirring, add dispensed quantity of Trisodium citrate dihydrate to step 1.1.6 purified water, ensure complete solubilisation.
1.1.8. Under stirring, add dispensed quantity of sodium chloride to step 1.1.7 purified water, ensure complete solubilisation.
1.1.9. Under stirring, add dispensed quantity of Disodium EDTA to step 1.1.9 purified water, ensure complete solubilisation.
1.1.10. Check pH and adjust the desire pH rang using Hydrochloric acid/Sodium hydroxide.
1.1.11. Filter step 1.1.10 solution using sterilizing grade filter.

Mixing of Polymeric Phase to API Phase:
1.1.12. Under stirring, transfer step 1.1.11 API solution to polymer solution of step 1.1.3 and continue stirring for 30 min. Record pH.

### Example 3: Ophthalmic Suspension of Methotrexate

Composition:

| | Example 3 |
|---|---|
| Name of Ingredient | % w/v |
| Methotrexate | 0.5% |
| Mannitol | 3.3% |
| Carbomer 974P | 0.46% |
| Tyloxapol | 0.3% |
| Sodium chloride | 0.25% |
| Disodium EDTA | 0.01% |
| Sodium hydroxide/ | Q.S to pH 7.2 |
| Hydrochloric acid | |
| Purified water | Q.S |

Process for Preparation:
1. Collect 100% batch size of purified water, purge with Nitrogen, Dissolved oxygen content should be less than 2 PPM, check pH.
2. Preparation of polymer phase:
2.1 Preparation of Vehicle Concentrate:
2.1.1 Collect 20% total batch size of purified water from step 1 into a clean SS vessel.
2.1.2 Under stirring add dispensed quantity of Mannitol, Sodium chloride, Disodium EDTA to step 2.1.1.
2.2 Preparation of Carbomer Slurry:
2.2.1 Collect 25% total batch size of purified water from step 1 into a clean SS vessel.
2.2.2 Under stirring add dispensed quantity of Carbomer 974P to step 2.2.1 in order to obtain Carbomer slurry.
2.3 Mixing of Vehicle Concentrate to Carbomer Slurry:
2.3.1 Under stirring to the Carbomer slurry in step 2.2.2, vehicle concentrate from step 2.1.2 was added and stirred.
2.3.2 pH of step 2.3.1 slurry was adjusted to 5.5, using 1 N Sodium hydroxide
2.3.3 Volume of the step 2.3.2 was made up to 60%.
3. Preparation of API Phase:
3.1 Collect 30% total batch size of purified water from step 1 into a clean SS vessel.
3.2 Under stirring add dispensed quantity of Tyloxapol followed by Methotrexate to step 3.1.
3.3 Step 3.2 API slurry was subjected to High Shear Mixing at 10,000 RPM for 15 min.
3.4 Volume of step 3.3 was made up to 40% using purified water from step 1.
3.5 Step 3.4 API slurry was subjected to size reduction using High pressure homogenizer at 15000 psi.
4. Mixing of API phase with polymer phase:
4.1 Under stirring step 3.5 API phase was added to step 2.3.3 polymer phase and stirred for 30 min to form the suspension of methotrexate.

### Example 4: Therapeutic Use of Methotrexate Ophthalmic Compositions of Example 1, 2 and 3 for Treatment of Anterior Uveitis in Rabbits

1. The Rabbits were divided into six groups with 5 animals in each group as normal control, induction group, reference group (aFolitrax-50 mg/2 ml), Example 1 formulation, Example 2 formulation and Example 3 formulation were subjected for treatment accordingly.
2. The rabbits were subjected to topical ophthalmic drop for the test formulations for a period of 12 days [50 μl of methotrexate solution of example 1 (50 μg of methotrexate), 50 μl of methotrexate solution of example 2 (250 μg of methotrexate) and 50 μl of methotrexate solution of example 3 (250 μg of methotrexate)] once a day in each eye, whereas reference group was treated subcutaneously on commencement of study (1 day).
3. All the groups except normal control groups were injected with 12.5 mg/kg of BSA (Bovine Serum Albumin) subcutaneously on Day-4, Day-6 and Day-8 (sensitization phase) of treatment. On Day 12 the rabbits sensitized with BSA was administered by intra-vitreal route with 2.5 mg/50 μL/animal as challenging phase.
4. The rabbits were observed for any abnormal inflammation for a period of 1 to 2 hours post intra-vitreal injection. Later, the rabbits were monitored and scored for the induction of flare and accumulation of inflammatory cells, causing opacity of cornea.
5. The scoring was made at initial (pre intra-vitreal injection) and 24 h post intra-vitreal injection for peak induction of anterior Uveitis conditions and recovery with treatment groups.
   *Note: Folitrax 50 mg/2 ml commercial available. 2.5 mg Human dose, Convert into animal dose, (2.5/60)×3.1=0.129 (0.13) mg/kg Rabbit dose.

End Point Scoring:

The scoring of the eyes under the slit lamp was made as per the below chart and final values were cumulated for the group.

| Score | Description for Flare Score |
|---|---|
| 0 | No Response |
| 1 | Mild: Clearly noticeable, Visible |
| 2 | Moderate: Without plastic aqueous |
| 3 | Marked: with plastic aqueous |
| 4 | Severe: Heavy with fibrin deposits |
| | and/or clots (iris details hazy) |

Results:

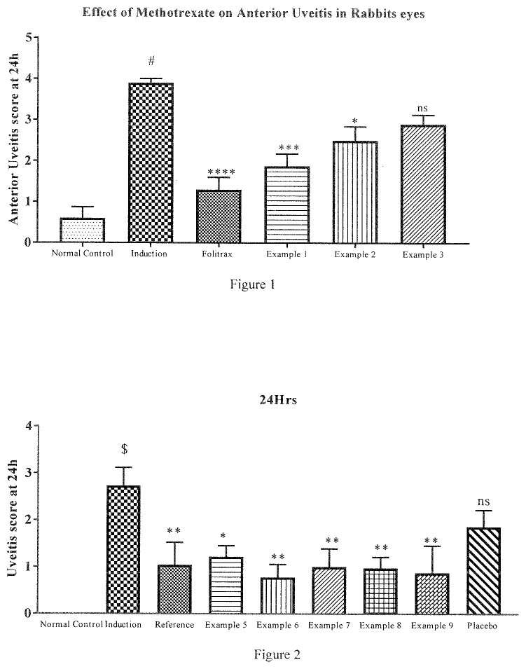

Summary of the anterior uveitis scoring at different time points post intra-vitreal BSA injection is disclosed in FIG. 1 (24 hours) and the results are depicted in Table 1.

TABLE 1

| | | | Anterior Uveitis | |
|---|---|---|---|---|
| S. | | | Scoring (Mean ± SEM) | |
| No | Group | Treatment | 0 hour | 24 hour |
| 1 | I | Normal Control | 0.00 ± 0.00 | 0.60 ± 0.27 |
| 2 | II | Induction | 0.00 ± 0.00 | 3.90 ± 0.10 |
| | | (Anterior | | |
| | | Uveitis) | | |
| 3 | III | Reference | 0.00 ± 0.00 | 1.30 ± 0.30 |
| | | (Folitrax) | | |
| 4 | IV | Example 1 | 0.00 ± 0.00 | 1.88 ± 0.30 |
| 5 | V | Example 2 | 0.00 ± 0.00 | 2.50 ± 0.34 |
| 6 | VI | Example 3 | 0.00 ± 0.00 | 2.60 ± 0.27 |

The scores of the individual animals are averaged and subjected to the One Way ANOVA followed by Dunnett's post hoc test for significance in the induction and recovery.

The Induction group produced a significant (p<0.0001) induction of Anterior Uveitis as compared to the Normal Control group.

The ophthalmic example 1 formulation with the dosage of 50 μg methotrexate once daily in Rabbit produced significant (p<0.001) prevention of anterior uveitis as compared to the induction group, whereas, example 2 formulation and example 3 formulation didn't produce any significant prevention of anterior uveitis.

The Reference group when treated subcutaneously produced significant (p<0.0001) prevention of Anterior Uveitis development as compared to induction group.

### Examples 5 to 9: Ophthalmic Solutions of Methotrexate

Composition:

| | Ex: 5 | Ex. 6 | Ex. 7 | Ex. 8 | Ex. 9 |
|---|---|---|---|---|---|
| Name of Ingredient | % w/v | % w/v | % w/v | % w/v | % w/v |
| Methotrexate | 0.01% | 0.025% | 0.05% | 0.1% | 0.25% |
| Tromethamine | 0.4% | 0.4% | 0.4% | 0.4% | 0.4% |
| Xanthan gum | 0.6% | 0.6% | 0.6% | 0.6% | 0.6% |
| Trisodium citrate | 0.2% | 0.2% | 0.2% | 0.2% | 0.2% |
| dihydrate | | | | | |
| Sodium chloride | 0.68% | 0.68% | 0.68% | 0.68% | 0.68% |
| Disodium EDTA | 0.025% | 0.025% | 0.025% | 0.025% | 0.025% |
| Benzalkonium chloride | 0.01% | 0.01% | 0.01% | 0.01% | 0.01% |
| Sodium hydroxide/ | Q.s to | Q.s to | Q.s to | Q.s to | Q.s to |
| hydrochloric acid | 7.2 | 6.8 | 7.2 | 7.2 | 7.2 |
| Purified water | Q.s | Q.s | Q.s | Q.s | Q.s |

Process for Preparation: The process for preparation is similar to examples 1 and 2 with the further addition of Benzalkonium chloride as the preservative, with the changes in the percentages of the methotrexate in the ophthalmic solution.

### Example 10: Therapeutic Use of Methotrexate of Examples 5, 6, 7, 8 and 9 in Rabbits for Treatment of Anterior Uveitis in Rabbits

1. The Rabbits were divided into nine groups with 5 animals in each group as normal control, induction group, reference group (Durezol), Example 5 formulation, Example 6 formulation, Example 7 formulation, Example 8 formulation, Example 9 formulation and Placebo formulation were subjected to treatment accordingly.
2. The rabbits were subjected to topical ophthalmic drop for the test formulations 50 μl of methotrexate solution of example 5 (5 μg of methotrexate), 50 μl of methotrexate solution of example 6 (12.5 μg of methotrexate), 50 μl of methotrexate solution of example 7 (25 μg of methotrexate), 50 μl of methotrexate solution of example 8 (50 μg of methotrexate), 50 μl of methotrexate solution of example 9 (125 μg of methotrexate) once a day in each eye; placebo group was administered with 50 μl of placebo formulation once a day each eye, whereas reference group was treated with 50 μl of Durezol® ophthalmic emulsion (Difluprednate 0.05% ophthalmic emulsion) once a day in each eye for a period of 12 days.
3. All the groups except normal control groups were injected with 12.5 mg/kg of BSA (Bovine Serum Albumin) subcutaneously on Day-4, Day-6 and Day-8 (sensitization phase) of treatment. On Day-12 the rabbits sensitized with BSA was administered by intra-vitreal route with 2.5 mg/5 μL/animal as challenging phase.
4. The rabbits were observed for any abnormal inflammation for a period of 1 to 2 hours post intra-vitreal injection. Later, the rabbits were monitored and scored for the induction of flare and accumulation of inflammatory cells, causing opacity of cornea.
5. The scoring was made at and 24 hours, 48 hours and 72 hours post intra-vitreal injection for peak induction of anterior uveitis conditions and recovery with treatment groups.

End Point Scoring:

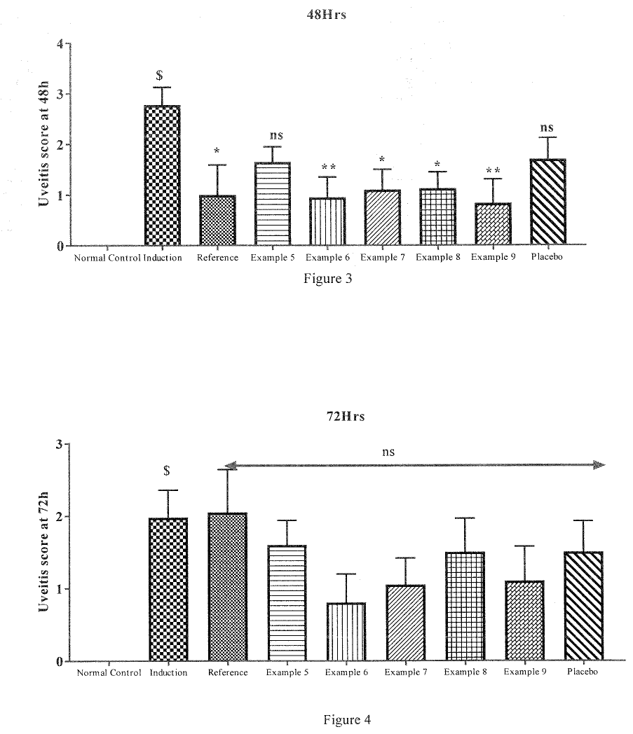

The scoring of the eyes was made at 24 hours, 48 hours and 72 hours post intra vitreal injection for peak induction of uveitis conditions. Scoring of the eyes under the slit lamp were made as per the below chart and final values were disclosed in Figures Table-2. FIGS. 2, 3 and 4 discloses the anterior uveitis activity of the results disclosed in Table-2 at 24 hours, 48 hours and 72 hours respectively post intra vitreal injection of RSA.

| Score | Description |
|---|---|
| 0 | No Response |
| 1 | Slight Congestion of iris vessels |
| 2 | Mild Congestion of iris vessels |
| 3 | Moderate Congestion of iris vessels |
| | and mild iris oedema |
| 4 | Marked congestion and dilation of iris |
| | vessels and moderate iris oedema. |

TABLE 2

| | | | | Anterior Uveitis | |
|---|---|---|---|---|---|
| S. | | | | Scoring (Mean ± SEM) | |
| No | Group | Treatment | 24 hour | 48 hour | 72 hour |
| 1 | I | Normal | 0.00 ± 0.00 | 0.00 ± 0.00 | 0.00 ± 0.00 |
| | | Control | | | |
| 2 | II | Induction | 2.7 ± 0.4 | 2.6 ± 0.4 | 2.0 ± 0.4 |
| | | (Anterior | | | |
| | | Uveitis) | | | |
| 3 | III | Reference | 1.5 ± 0.5 | 1.9 ± 0.6 | 2.1 ± 0.6 |
| | | (Durezol) | | | |
| 4 | IV | Example 5 | 1.5 ± 0.3 | 1.7 ± 0.3 | 1.6 ± 0.3 |
| 5 | V | Example 6 | 1.1 ± 0.4 | 1.0 ± 0.4 | 0.8 ± 0.4 |
| 6 | VI | Example 7 | 1.0 ± 0.4 | 1.1 ± 0.4 | 1.1 ± 0.4 |
| 7 | VII | Example 8 | 1.2 ± 0.3 | 1.7 ± 0.4 | 1.5 ± 0.5 |
| 8 | VIII | Example 9 | 1.4 ± 0.6 | 1.2 ± 0.5 | 1.1 ± 0.5 |
| 9 | IX | Placebo | 1.9 ± 0.4 | 1.7 ± 0.4 | 1.5 ± 0.4 |

The Induction group produced a significant (p<0.0001) induction of Anterior Uveitis as compared to the Normal Control group.

The ophthalmic example 6 formulation with the dosage of 12.5 μg methotrexate administered once daily in each eye of Rabbit produced significant (p<0.001) prevention of Anterior Uveitis at 24 hours and 48 hours.

### Examples 11 to 14: Ophthalmic Solutions of Methotrexate

Composition:

| | Ex: 11 | Ex. 12 | Ex. 13 | Ex. 14 |
|---|---|---|---|---|
| Name of Ingredient | % w/v | % w/v | % w/v | % w/v |
| Methotrexate | 0.025% | 0.025% | 0.025% | 0.1% |
| Tromethamine | 0.05% | 0.2% | 0.8% | 1.4% |
| Xanthan gum | 0.6% | 0.6% | 0.6% | 0.6% |
| Trisodium citrate dihydrate | 0.2% | 0.2% | 0.2% | 0.2% |
| Sodium chloride | 0.68% | 0.68% | 0.68% | 0.68% |
| Disodium EDTA | 0.025% | 0.025% | 0.025% | 0.025% |
| Benzalkonium chloride | 0.01% | 0.01% | 0.01% | 0.01% |
| Sodium hydroxide/ | Q.s to | Q.s to | Q.s to | Q.s to |
| hydrochloric acid | 6.8 | 6.8 | 6.8 | 6.8 |
| Purified water | Q.s | Q.s | Q.s | Q.s |

Process for Preparation: The process for preparation is similar to examples 6 with the changes in the percentages of the tromethamine in the ophthalmic solution.

### Examples 15: Stability Studies

The compositions prepared as in examples 6, 11, 12, 13 and 14 are stored at 2° C.-8° C. and was tested for impurities at specific intervals. The results of examples 6, 11, 12, 13 and 14 are as tabulated in tables 3 to 7 respectively.

TABLE 3

| | | Unknown | Single | |
|---|---|---|---|---|
| | | impurity | maximum | Total |
| Time | Assay | at 0.68 | unknown | Impurity |
| Initial | 97.5 | 0.25 | 0.08 | 0.50 |
| 1 M | 99.0 | 0.48 | 0.07 | 0.76 |
| 3 M | 98.3 | 0.51 | 0.08 | 0.74 |
| 6 M | 102.0 | 0.55 | 0.10 | 0.83 |

TABLE 4

| | | Unknown | Single | |
|---|---|---|---|---|
| | | impurity | maximum | Total |
| Time | Assay | at 0.68 | unknown | Impurity |
| Initial | 100.6 | 0.22 | 0.09 | 0.49 |
| 1 M | 98.7 | 1.10 | 0.09 | 1.59 |
| 3 M | 97.4 | 2.75 | 0.09 | 3.18 |
| 6 M | 95.5 | 5.75 | 0.08 | 6.15 |

TABLE 5

| | | Unknown | Single | |
|---|---|---|---|---|
| | | impurity | maximum | Total |
| Time | Assay | at 0.68 | unknown | Impurity |
| Initial | 98.2 | 0.25 | 0.08 | 0.50 |
| 1 M | 98.2 | 0.25 | 0.07 | 0.52 |
| 3 M | 97.9 | 0.27 | 0.07 | 0.48 |
| 6 M | 100.5 | 0.31 | 0.11 | 0.60 |

TABLE 6

| | | Unknown | Single | |
|---|---|---|---|---|
| | | impurity | maximum | Total |
| Time | Assay | at 0.68 | unknown | Impurity |
| Initial | 100.4 | 0.28 | 0.07 | 0.48 |
| 1 M | 99.6 | 0.35 | 0.11 | 0.65 |
| 3 M | 98.4 | 0.72 | 0.10 | 1.23 |
| 6 M | 97.6 | 1.04 | 0.11 | 1.94 |

TABLE 7

| | | Unknown | Single | |
|---|---|---|---|---|
| | | impurity at | maximum | Total |
| Time | Assay | 0.68 | unknown | Impurity |
| Initial | 100.2 | 0.25 | 0.08 | 0.50 |
| 1 M | 99.4 | 1.06 | 0.08 | 1.36 |
| 3 M | 97.6 | 2.77 | 0.07 | 3.08 |
| 6 M | 94.3 | 5.97 | 0.08 | 6.53 |

1. A method of treating anterior uveitis of the mammalian eye, said method-comprising administering to the mammal in eye about 5 μg to about 200 μg of methotrexate.

2. A method of claim 1, wherein methotrexate is administered as an ophthalmic solution.

3. A method of claim 1, wherein the methotrexate is administered once a day, twice a day or thrice a day in each affected eye.

4. A method of treating anterior uveitis of the mammalian eye, said method comprising administering to the mammal in eye a methotrexate topical ophthalmic composition comprising about 0.01% w/v to about 0.5% w/v methotrexate.

5. A method of claim 4, wherein the composition comprises about 0.01% w/v to about 0.1% w/v methotrexate.

6. A method of claim 5, wherein the composition comprises about 0.025% w/v methotrexate.

7. A method of claim 4, wherein the composition comprises about 0.01% w/v to about 0.05% w/v of methotrexate, about 0.2% w/v to about 0.8% w/v of solubilizers, about 0.1% w/v to about 1.0% w/v of viscosifying agents and pH adjusting agents; and wherein the composition has the pH of about 6.8 to about 8.5.

8. A method of claim 4, wherein the composition comprises about 0.01% w/v to about 0.05% w/v of methotrexate, about 0.2% w/v to about 0.8% w/v of tromethamine, about 0.1% w/v to about 1.0% w/v of xanthan gum and pH adjusting agents; and wherein the composition has the pH of about 6.8 to about 8.5.

9. A method of claim 4, wherein the composition comprises about 0.025% w/v of methotrexate, about 0.4% w/v of tromethamine, about 0.6% w/v of xanthan gum and pH adjusting agents; and wherein the composition has the pH of about 6.8 to about 8.5.

10. A method of claim 4, wherein the composition consists essentially of about 0.025% w/v of methotrexate, about 0.4% w/v of tromethamine, about 0.6% w/v of xanthan gum, buffering agents, osmotic agents, antioxidants, preservatives and pH adjusting agents; and wherein the composition has the pH of about 6.8 to about 8.5.
